(19) United States
(12) Patent Application Publication
(10) Pub. No.: US 2022/0272971 A1
(43) Pub. Date: Sep. 1, 2022

(54) COMPOSITIONS, MEANS AND METHODS FOR A NOVEL INSECT REPELLENT

(71) Applicant: Yosef LOMNICKY, Givat Shmuel (IL)

(72) Inventor: Yosef LOMNICKY, Givat Shmuel (IL)

(21) Appl. No.: 17/625,752

(22) PCT Filed: Jul. 8, 2020
(86) PCT No.: PCT/IL2020/050764

§ 371 Date: Jan. 9, 2022

(30) Foreign Application Priority Data
Jul. 9, 2019 (IL) 267941

(51) Int. Cl. A01N 35/02; A01P 17/00; A01N 43/32; A01N 65/26; A61K 36/886; A61K 36/61; A61K 36/534; A61K 36/73; A01P 7/04; A01N 25/34; A01M 1/20; A01M 29/12

(52) U.S. Cl. CPC A01N 35/02; A01P 17/00; A01N 43/32; A01N 65/26; A61K 36/886; A61K 36/61; A61K 36/534; A61K 36/73; A01P 7/04; A01N 25/34; A01M 1/2022; A01M 29/12

(57) ABSTRACT

An insect repellent composition for repelling insects from mammals and vegetation. This composition comprises: dihydroxyacetone (DHA) and a carrier suitable for insect repellent compositions. DHA is the active ingredient and the composition is efficacious in repelling insects.

### FIELD OF INVENTION

The present invention relates to the field of insect repellency, and a repellent material.

### BACKGROUND OF INVENTION

In recent years, there has been an increased interest in insect repellents, due to periodic outbreaks of mosquito-borne diseases, such as the Zika virus, the West-Nile virus and others. The most commonly used insect repellent today is the synthetic compound, DEET (N,N-Diethyl-meta-toluamide). However, DEET may cause skin and eye irritation. DEET also dissolves most plastics, as well as rayon and spandex, and could thus cause damage to clothing, sunglasses, watchstraps and the like. Another minor drawback is its unpleasant odor. Furthermore, there has been some controversy regarding the safety of DEET, and exposure to high doses may induce neurological damage [1-3]. Plant-based insect repellents are generally considered safer; however, they have not been as rigorously tested as DEET and other synthetic insect repellents, and side-effects such as contact dermatitis, as well as more serious neurotoxic effects of many of these natural repellents have been reported [4]. In addition, most of these plant-based insect repellents are quite volatile, and do not provide long-lasting protection (60-180 min.).

In light of the above, there is a need for a long-acting, effective and non-toxic insect repellent.

### SUMMARY OF THE INVENTION

The present invention relates to the field of insect repellency, and more specifically, to a new insect repellent compound.

It is an object of this invention to provide an insect repellent composition for repelling insects from mammals and vegetation, comprising: dihydroxyacetone (DHA) and a carrier suitable for insect repellent compositions, with DHA as the active ingredient.

It is another object of this invention to provide an insect repellent composition for repelling insects from mammals, wherein the mammals are humans or animals.

It is also an object of this invention to provide an insect repellent composition for repelling insects from vegetation, wherein said plants are tobacco, cannabis, or organically grown fruits and vegetables for human and animal consumption.

It is also an object of this invention to provide an insect repellent composition, wherein the concentration of DHA is in the range of 0.5% to 50% (% weight).

It is another object of this invention to provide a composition for insect repellency, wherein this composition is in a form selected from a group consisting of liquid, aerosol, lotion, cream, gel, foam, shampoo, solid and any combination thereof.

It is another object of this invention to provide an insect repellent composition, wherein the composition is suitable for topical use.

It is a further object of this invention to provide an insect repellent composition, wherein the composition is suitable for spatial use.

It is another object of this invention to provide a composition for insect repellency wherein the insects to be repelled are selected from a group consisting of: mosquitos, fleas, ants, sand flies, spiders, aphids, tobacco moths and head-lice.

It is also an object of this invention to provide an insect repellent composition, wherein the composition additionally comprises at least one compound selected from the group consisting of water, ethanol, emulsifiers, preservatives, emollients, humectants, oils, surfactants, waxes, solvents, rheology modifiers, suspending agents, thickeners, oils, buffering agents, chelating agents, pH adjusters, chelates, perfumes, brighteners, and any combination thereof.

It is also an object of this invention to provide a composition for insect repellency, wherein the composition further comprises a Maillard reaction inhibitor, selected from a group consisting of aminoguanidine, amphotericin B, plant polyphenols, pyridoxamine, carnosine, thiamine pyrophosphate, niacin, and any combination thereof.

It is another object of this invention to provide a composition for insect repellency formulated for application on clothing, hats, curtains, nets, bed-sheets, wipes, adhesive patches, hair bows, hairpins, jewelry or baby bracelets.

It is also an object of this invention to provide a composition for insect repellency, wherein the composition is suitable for application to adults, children or babies.

It is a further object of this invention to provide a composition for insect repellency, wherein the composition is in a form of immediate release or slow release, or a combination thereof.

It is also an object of this invention to provide a slow release composition for insect repellency, wherein the slow release composition comprises bifunctional DHA esters with short-chain carboxylic acids, and said esters are hydrolysable to form DHA.

It is a further object of this invention to provide bifunctional DHA esters wherein the short chain carboxylic acids have a carbon backbone of 4 to 12 carbon atoms.

It is also an object of this invention to provide bifunctional DHA esters for insect repellency, wherein the repelling effect of DHA and the short-chain carboxylic acids combination is a synergetic effect.

It is a further object of this invention to provide a composition for slow release insect repellents, wherein the slow release formulation comprises DHA incorporation in a polymeric matrix.

It is also an object of this invention to provide a composition for slow release insect repellents, wherein the slow release formulation comprises micro-encapsulation of DHA.

It is also an object of this invention to provide a composition for slow release insect repellents, wherein the slow release formulation comprises DHA incorporated into liposomes.

It is another object of this invention to provide a DHA insect repellent composition, wherein the composition further comprises at least one additional insect repellent, selected from a group consisting of menthol, geraniol, thyme oil, lemongrass oil, rosemary oil jasmine, lemon eucalyptus and citronella extracts, and any combination thereof.

It is another object of this invention to provide a DHA insect repellent composition, wherein the composition further comprises at least one insect bite soother, selected from a group consisting of Aloe Vera, tea tree essential oil, peppermint extract, apple cider vinegar, or any combination thereof.

It is another object of this invention to provide a DHA insect repellent composition, wherein the composition further comprises at least one additional pesticide, selected from a group consisting of azadirachin, pyrethrins, neem oil, fatty acid potassium salts.

It is also an object of this invention to provide a method of repelling insects, consisting of an insect repellent composition, and application of the composition to a subject, thereby repelling insects.

It is a further object of this invention to provide a kit useful for repelling insects, comprising of a vaporizer, an insect repellent composition and instructions for use.

It is also an object of this invention to provide a kit useful for insect repellency, wherein the insect repellent composition is liquid.

It is also an object of this invention to provide a kit useful for insect repellency, wherein the insect repellent composition is solid.

It is another object of this invention to provide a kit useful for insect repellency, wherein the vaporizer is a plug-in electrical device.

It is another object of this invention to provide a kit useful for insect repellency, wherein the vaporizer is a spraying device.

It is also an object of this invention to provide a kit useful for insect repellency, wherein the vaporizer is a coil.

It is a further object of this invention to provide an insect repellent composition for repelling insects from mammals and vegetation, comprising: a dihydroxyacetone (DHA) derivative and a carrier suitable for insect repellent compositions, wherein the DHA derivative is the active ingredient.

### BRIEF DESCRIPTION OF THE DRAWING

The accompanying drawing, which is included to provide a further understanding of the invention, and is incorporated in, and constitutes a part of this specification, illustrates an embodiment of the invention and together with the description serves to explain the principles of the invention

FIG. 1: Repellency of control, 20% DHA, and commercial Sano insect repellent with a composition of 20% of citrepel 75 dissolved in water, against the common mosquito, Culex quinquefasciatus. Data are presented as mean±SEM.

### DETAILED DESCRIPTION OF THE INVENTION

The present invention discloses a compound used for providing protection against insect bites; in particular, mosquitos, fleas, ants, sand flies, spiders and head-lice, as well as acting as an insect repellent against aphids and tobacco moths. The inventors have surprisingly found that dihydroxyacetone (DHA), is active as an insect repellent. Protection against insects is thus provided by dihydroxyacetone (DHA), in various compositions. Methods for insect repellency include both topical and spatial compositions.

As used hereinafter, the term “repellent” generally refers to preventing biting by insects.

As used hereinafter, the term “active ingredient” refers to the substance(s) in a composition, which provide the desired effect.

As used hereinafter, the term “efficacious in repelling insects” refers to the characteristics of a composition providing the same protection as a commercial insect repellent.

As used hereinafter, the term “topical” generally refers to compositions applied directly to the skin or plant.

As used hereinafter, the term “spatial repellents” generally refers to chemicals which are released into the air of the treated space, resulting in the prevention or reduction of insect biting.

As used hereinafter, the term “insect” generally refers to mosquitos, fleas, ants, sand flies, and spiders, aphids, tobacco moths and head-lice.

As used hereinafter, the term “Dihydroxyacetone” (DHA) generally refers to the following compound:

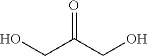

An earlier attempt to provide long-lasting insect repellants was reported by Quintana et al (U.S. Pat. No. 3,668,226). Mono-esters of dihydroxyacetone (DHA) with lower aliphatic carboxylic acids, exhibited long-lasting insect repellent activity. However, the insect repelling effect was attributed to the aliphatic carboxylic acids, while the DHA moiety was utilized as a dermal anchoring moiety, thus providing the longer-lasting insect repelling effect of these aliphatic carboxylic acids.

Dihydroxyacetone (DHA) itself has been used for many years in the cosmetics industry, as the active ingredient of sunless-tanning products and has been approved by the FDA as a cosmetic ingredient since 1977. The skin-browning effect is a result of a Maillard reaction between DHA and the amino acids of keratin, the major component of the skin surface. This effect fades after 3-10 days following application, in the course of mechanical abrasion or natural exfoliation of the skin. DHA is not systemically absorbed, is non-toxic, and its phosphate ester (DHAP) is involved in many metabolic pathways, such as glycolysis and the Calvin cycle. In response to concern regarding the safety of DHA application to skin, a report by the European Commissions' Scientific Committee on Consumer Safety (SCCS), issued in 2010, concluded that DHA sprays, in concentrations of up to 10%, are safe to use. DHA was also found to be non-irritant, non-carcinogenic and did not cause skin sensitization or photo sensitization effects (4).

Topical formations of insect repellents include creams, ointments, gels and sprays. Another method for topical formulations include alcohol-based foams (U.S. Pat. No. 7,683,018 B2, to Koivisto et al.; US patent 20130165530 A1, to Hillman et al). The use of wipes containing insect repellents is another method for topical application of insect repellents, such as reported by Fellows (WO 1989003639 A1) and Glass (AU patent 2009100380 A4). Additional methods for topical application of insect repellents include adhesive patches, insect repellent-containing bracelets and jewelry, as shown, for example by Quin, (US patent 20130095162 A1). Bracelets containing insect repellent formulations suitable for use by babies and toddlers are also marketed. Insect repellent collars for dogs and horses provide another means for insect repellency (US patent 20120315317 A1)

Another factor to consider when formulating the product is the vaporization rate of the compound. The insect repellent must evaporate from the skin in order to work. High evaporation rates lead to short-term repellency, such as the case with many plant-based repellents. Slow release formulations provide prolonged action of the repellent, as well as reduce toxicity due to lower concentrations of the active ingredient on the skin. For example, a mixture of short-chain carboxylic acids (mainly C₈ and C₉), with different degrees of volatility has been reported to possess long-term insect repellency (U.S. Pat. No. 6,444,216). Esters of such insect repellent compounds necessitate hydrolysis prior to release of the active compound, thereby providing a long-lasting effect. DHA esters of such compounds provide a synergistic effect, due to the repellent effect of both components.

Examples of slow release formulations include micro-encapsulation in synthetic and natural polymers, an example of which is the slow release of DHA enclosed in a dimer form in a watertight composition which releases the DHA when applied (U.S. Pat. No. 5,458,872 A). Other slow release formulations include solid lipid micro and nano particles, liposomes, and complexation with cyclodextrines (Tavares et al., 2018).

Slow release formulations of DHA may also be used for the coating of fabrics, such as nets, bed-sheets, etc. Slow release formulations are more effective for fabrics, since most insect repellents are volatile, and do not provide long-lasting protection.

In general, spatial repellent compounds are highly volatile and capable of diffusing through the air in treated regions. The volatilization of these compounds creates repellent vapors that deter insects. Methods used for spatial insect repellency include the use of plugged in devices, which heat and vaporize the insect repellent, burning candles, such as citronella candles, burning coils containing insect repellents, and the use of an electrostatic spraying device for insect repellents has been reported by Hadingham et al (WO 2012096117 A1)

Liquid formulations of DHA may include additives commonly used in cosmetics, such as emulsifiers, dispersing agents, and film-forming agents. Examples of emulsifiers and dispersing agents usable in the present invention include soap (stearic acid), ethoxylated fatty alcohols, PEG esters of fatty acids. Examples of film forming agents include nitrocellulose and acetylcellulose. Additional suitable formulations include creams, lotions, gels, foams and sprays, with additives commonly used in such cosmetic formulations.

The following examples are representative of some formulations that may be based on the above disclosure. Those skilled in the art will recognize other various formulations and materials that are effective in achieving the invention disclosed herein.

### Example 1

In an embodiment of this invention, the insect repellent effect of a 20% DHA solution was studied. These studies were carried out at Poseidon Sciences Insect Control Laboratory (Miagao, Iloilo, Philippines), a breeding facility for C. quinquefasciatus, for research and development. The insect repellency of a DHA solution, comprising 20% DHA dissolved in a 30% ethanol aqueous solution, was compared to that of a Placebo solution, comprising 30% ethanol in water, as a negative control solution. As a positive control, the repellency of DHA was also compared to that of a commercial Sano insect repellent, a plant based repellent, using a composition of 20% of citrepel 75 dissolved in water. The placebo and the 20% DHA solution were kept in a refrigerator until the day of use.

The samples were removed from the refrigerator and kept at room temperature for 3 hours before the tests were undertaken.

The above-mentioned solutions were then tested on a total of 10 subjects (age 19 to 44 comprising a total of 6 women and 4 men), all in good health. Each subject had signed an Informed Consent Form. This study had been approved by Poseidon Sciences IRB (Institutional Review Board). The study group included 3 women and two men subjects. The Control Group included 3 women and 2 men subjects who received placebo treatment.

All studies were conducted starting at 4:00 PM and all human subjects did not consume any food for a period of at least 2 hours prior to the repellent test. All subjects did not use any cosmetic product for at least 24 hours prior to the tests. Four drops of the DHA solution or placebo solution were applied on the exposed forearm and immediately spread over the exposed skin with a gloved finger. As requested, a period of 1 minute was allowed to lapse before insertion of the forearm inside the cage. The study was conducted at room temperature (27° C. to 29° C.) under ambient light conditions.

Following application of the tested solution to the arm of the subject, testing was carried out as follows: a research scientist recorded the time of insertion of the arm into the cage and observed mosquito landing. The subject indicated that a confirmed bite had occurred and the investigator confirmed by visual examination of the forearm. This was further reconfirmed by a second bite before terminating the study. The Protection Time, defined in this study as the period that had elapsed from the insertion of the arm into the cage and the time for the first confirmed bite (indicating that the tested product had reached its limit of protection from mosquitoes), was recorded for each subject. Results are shown in Table 1 below, presented for each subject, as well as the mean±SD and SEM for each group.

TABLE 1
Repellent effects of placebo, 20% DHA, and Sano repellent,
against the mosquito Culex quinquefasciatus,

| Product | Protection Time (minutes) | | | | | | | |
|---|---|---|---|---|---|---|---|---|
| Name | Subject 1 | Subject 2 | Subject 3 | Subject 4 | Subject 5 | Mean | SD | SEM |
| Placebo | 0.17 | 0.13 | 0.17 | 0.12 | 0.20 | 0.16 | 0.03 | 0.01 |
| 20% | 45 | 50 | 40 | 35 | 56 | 45.2 | 7.36 | 3.35 |
| DHA | | | | | | | | |
| Sano ™ | 45 | 60 | 70 | 50 | 60 | 57.0 | 9.8 | 4.50 |
| Repellent | | | | | | | | |

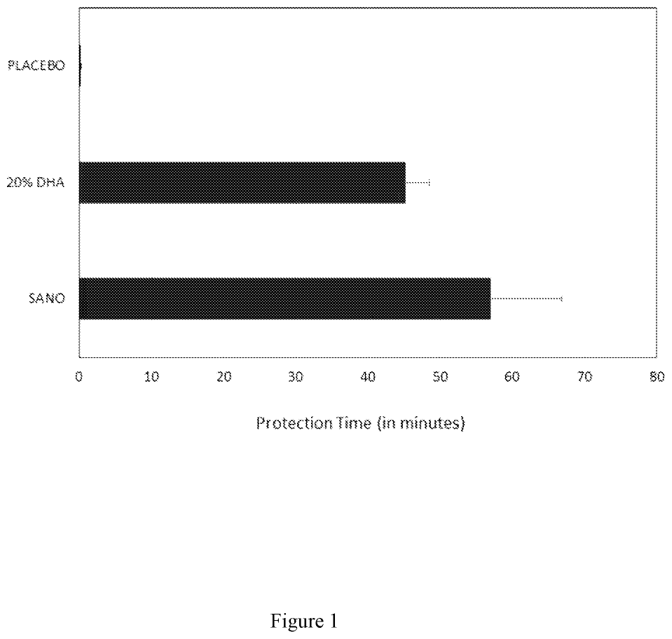

The data obtained from these subjects is also presented in FIG. 1. Data is presented as mean±SEM. Data analysis (using a Student t-test) shows that the repellency of 20% DHA is significantly different from that of the placebo (P<0.0001), but no significant difference between 20% DHA and the commercial Sano product was observed (P<0.064). It should be noted that data from the placebo and 20% DHA were obtained at a different time from the data from the SANO product, and results are presented here as an historical comparison. Thus, the mean protection time found for DHA (45 minutes) is in the range of that of competing natural products (30 minutes-1 hour), tested under the conditions of this bioassay.

### Example 2

In another preferred embodiment of this invention, compositions of DHA are prepared for topical application. DHA compositions according to this invention include creams, ointments, lotions and gels.

A non-limiting example for a composition suitable for a cream, containing 20% DHA is shown in Table 2.

TABLE 2
Composition of a 20% DHA repellent cream

| Ingredient | % of weight |
|---|---|
| Water | 76.5 |
| DHA | 20 |
| Sodium Lauryl Sulfate | 5 |
| Cocamidopropyl betaine | 5 |
| PEG 8 | 1.5 |
| Glycerine | 1 |
| Disodium Lauryl Sulfosuccinate | 1 |

Another non-limiting example for a DHA composition according to this invention includes alco-gels, used for hand and skin sanitization. Table 3 shows a composition suitable for a DHA containing alco-gel.

TABLE 3
Composition of a DHA containing alco-gel

| Ingredient | % of weight |
|---|---|
| Water | 18.7 |
| DHA | 10 |
| Carbomer | 0.24 |
| Glycerine | 0.7 |
| Ethanol | 70 |
| Triisopropanolamine | 0.26 |
| Fragrance | 0.1 |

Additional topical compositions of DHA include inter alia sprays, aerosols and foams. All such compositions are prepared by adding suitable carriers and/or additives which are conventional in cosmetics. A non-limiting example for a formulation of a DHA-containing insect repellent spray is shown in table 4.

TABLE 4
Composition of DHA insect repellent spray

| Ingredient | % weight |
|---|---|
| Water | 50.5 |
| DHA | 15 |
| Ethanol | 25 |
| hexane | 1.5 |
| Polysorbate 80 | 8 |

Topical DHA formulations according to this invention, also include inter alia formulations suitable for babies, such as baby creams, lotions, and baby oils. A non-limiting example for a formulation of DHA-containing baby cream is shown in Table 5.

TABLE 5
Composition of DHA containing baby cream

| Ingredients | % weight |
|---|---|
| Water | 72.8 |
| DHA | 5 |
| Glycerol monostearate | 2.5 |
| Stearic acid | 1.2 |
| Lanolin | 1.0 |
| Technical white oil | 15 |
| Glycerol | 2.5 |
| Triethanolamine | A few drops |

### Example 3

In another preferred embodiment of this invention, slow-release formulations of DHA are used for insect repellency. In one example, slow release is obtained by encapsulation of DHA in microcapsules. Examples of materials used for microencapsulation include, but are not limited to, sodium alginate, gelatin, ethyl cellulose and polyurethane. In another example of a slow release formulation, DHA is encapsulated in solid lipid micro or nanoparticles. Encapsulation of DHA in liposomes, formulated from phospholipids, provides an additional formulation of slow release of DHA.

### Example 4

In another preferred embodiment of this invention, DHA is impregnated into fabrics, such as clothing bed sheets, mosquito netting, curtains, gloves, hats etc. Clothing according to this invention includes out-door clothing, as well as pajamas. Impregnation is carried out using various techniques, such as soaking the fabric in a solution containing DHA, or spraying it onto the fabric, using a DHA spray composition suitable for fabrics. Such DHA compositions may also be used, according to this invention, for application to backpacks, tents, adhesive strips to be applied to clothing, as well as wearable wrist and ankle bracelets.

DHA may also be applied to the fabric as a slow release formulation, comprising encapsulation of DHA in microcapsules, using materials known in the art for microencapsulation, with the microcapsule consisting of natural or synthetic polymers. DHA may also be impregnated in wipes, and adhesive patches, the latter providing a slow release formulation of DHA.

### Example 5

Another preferred embodiment of this invention includes kits for spatial insect repellent release. A kit for spatial release of DHA, according to this invention, includes a plugged-in electrical device, for heating and evaporating DHA. DHA formulations suitable for such devices include tablets and liquid solutions. Liquid solutions contain 0.5-50% DHA, dissolved in hydrocarbon organic solvents. Tablet formulations contain 4-50% DHA (DHA powder or liquid can be used at relatively high concentrations in devices or patches). Another example is a spatial release kit for DHA comprising a spraying device and a dispersion medium for DHA, thereby releasing DHA into the room. Such devices are suitable for indoor use. Spatial devices for outside use include candles and burned coils.

### Example 6

In another embodiment of this invention, inhibitors of the Maillard reaction are added to the DHA topical compositions. Such inhibitors prevent the skin-coloring effect of DHA. These inhibitors include amino-guanidine and amphoterin B, as well as naturally occurring compounds such as plant polyphenols, pyridoxamine (a vitamin B6 derivative), carnosine (an anti-oxidant), thiamine pyrophosphate (a vitamin B₁ derivative), and niacin (a form of vitamin B₃).

### Example 7

Short-chain carboxylic acids (C₄-C₁₂) have been shown to exhibit insect repellent activity (Reifenrath, in U.S. Pat. No. 6,444,216 B2). The synthesis of DHA monoesters of such carboxylic acids has been reported (Quintana et al, U.S. Pat. No. 3,668,226 A). Following topical application of such DHA monoesters with such short chain carboxylic acids, these esters then react with agents found on the skin, resulting in the gradual release of DHA and the short-chain carboxylic acids. Since both components exhibit insect repellent activity, a synergistic effect is obtained. Other derivatives of DHA, include di-substituted derivatives, of the general formula:

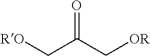

Where R and R″ are short chain carboxylic acids, with a C₄-C₁₂ carbon backbone, and may be the same or different. Hydrolysis of these compounds following topical application, results in the release of DHA and these short-chain carboxylic acids, resulting in a synergetic effect.

### Example 8

In another embodiment of this invention, DHA is combined with other insect repellents. Such combinations have been shown to be more effective than each component alone. These additional insect repellents may be plant based insect repellents, such as menthol, geraniol, thyme oil, lemongrass oil, rosemary oil jasmine, and citronella extracts. DHA may be combined with one or more of these natural repellents. In these compositions, DHA may be used in concentrations of 5%-50%, and the plant based components in concentrations of 0.5%-50%. These combinations are used with suitable formulations. DHA may also be used in combination with low concentrations of DEET, thus avoiding side effects of DEET when used in high concentrations. Combinations of DHA with DEET include DHA in concentrations of 0.5%-50%, and DEET in concentrations of 1%-10%, in a suitable formulation. DHA may also be used in combination with other components used for relief from itchiness caused by insect bites. Such components include, but are not limited to aloe Vera, tea tree essential oil, peppermint extract and apple cider vinegar.

### Example 9

In another preferred embodiment of this invention, DHA is used for repelling insects from plants. The insects repelled by DHA include aphids and tobacco moths. DHA can be used in concentrations of 0.5%-50%. DHA may be also used in combination with additional plant based and EPA-approved pesticides. Such pesticides include azadirachtin, pyrethrins, Neem oil, and fatty acid potassium salts. These additional pesticides are used in concentrations of 0.1%-1.0%

### Example 10

In another preferred embodiment of this invention, DHA is used in lice repellent compositions. Alcohols, such as isopropyl alcohol, have been shown to be effective anti-lice agents (GB 1,604,857; EP 0262885). DHA may be used in concentrations of 5%-20%. A non-limiting example for formulation of a DHA-containing lice repellent hairspray is shown in Table 6.

TABLE 6
Composition of DHA lice repellent hairspray

| Ingredient | % weight |
|---|---|
| Polyvinypyrrolidone/acrylate copolymer | 5 |
| Aminomethylpropanol (AMP) | 0.23 |
| DHA | 5 |
| Pyridoxamine | 5 |
| Propane/Butane 25:75 | 50 |
| Perfume | qs (as needed) |
| Ethanol (abs.) | Balance |

1. An insect repellent composition for repelling insects from mammals and plants, comprising:
a. Dihydroxyacetone (DHA); and
b. a carrier suitable for insect repellent compositions,
wherein DHA is the active ingredient and said composition is efficacious in repelling insects.

2. The composition of claim 1, wherein said mammals are humans or animals.

3. The composition of claim 1 wherein said plants are selected from a group consisting of: tobacco, cannabis, or organically grown fruits and vegetables for human and animal consumption.

4. The composition of claim 1, wherein the concentration of DHA is in the range of 0.5% to 50% (% wt)

5. The composition of claim 1, wherein said composition is in a form selected from a group consisting of liquid, aerosol, lotion, cream, gel, foam, shampoo, solid and any combination thereof.

6. The composition of claim 1, wherein said composition is suitable for topical use.

7. The composition of claim 1, wherein said composition is suitable for spatial use.

8. The composition of claim 1, wherein said insects comprise at least one selected from a group consisting of: mosquitos, fleas, ants, sand flies, spiders, aphids, tobacco moths and head-lice.

9. The composition of claim 1, wherein said composition additionally comprises at least one compound selected from the group consisting of water, ethanol, emulsifiers, preservatives, emollients, humectants, oils, surfactants, waxes, solvents, rheology modifiers, suspending agents, thickeners, oils, buffering agents, chelating agents, pH adjusters, chelates, perfumes, brighteners, and any combination thereof.

10. The composition of claim 1, further comprising a Maillard reaction inhibitor, selected from a group consisting of aminoguanidine, amphotericin B, plant polyphenols, pyridoxamine, carnosine, thiamine pyrophosphate niacin, and any combination thereof.

11. The composition of claim 1, formulated for application on clothing, hats, curtains, nets, bed-sheets, wipes, adhesive patches, hair bows, hairpins, jewelry or baby bracelets and is suitable for application to adults, children or babies.

12. The composition of claim 1, wherein said composition is in a form of immediate release or slow release, or a combination thereof.

13. The composition of claim 13, wherein said slow release formulation comprises DHA incorporation in a polymeric matrix.

14. The composition of claim 13, wherein said slow release formulation comprises micro-encapsulation of DHA or DHA incorporated into liposomes.

15. The composition of claim 1, further comprising at least one insect bite soother, selected from a group consisting of Aloe Vera, tea tree essential oil, peppermint extract, apple cider vinegar, or any combination thereof.

16. The composition of claim 1, further comprising at least one additional pesticide, selected from a group consisting of azadirachin, pyrethrins, neem oil, fatty acid potassium salts.

17. A method of repelling insects, comprising:
a. obtaining the composition of claim 1, and
b. applying said composition to a subject thereby repelling insects.

18. A kit useful for repelling insects comprising
a. a vaporizer
b. the composition of claim 1
c. instructions for use.

19. The kit of claim 20, wherein said composition is liquid or solid

20. The kit of claim 20, wherein the vaporizer is at least one selected from the group consisting of a plug-in electrical device, a spraying device or a coil.
